(19) United States
(12) Patent Application Publication
(10) Pub. No.: US 2023/0277598 A1
(43) Pub. Date: Sep. 7, 2023

(54) TREATMENT OF TRAUMA ASSOCIATED COGNITIVE DYSFUNCTION USING MESENCHYMAL STEM CELL APOPTOTIC BODIES AND COMPOSITIONS THEREOF

(71) Applicant: Therapeutic Solutions International, Inc., Oceanside, CA (US)

(72) Inventors: Santosh KESARI, Los Angeles, CA (US); Thomas ICHIM, San Diego, CA (US); Timothy Dixon, Oceanside, CA (US); Kalina O'CONNOR, Bonita, CA (US); Wais KAIHANI, San Diego, CA (US); Duncan HUNTER, Alpine, CA (US)

(73) Assignee: Therapeutic Solutions International, Inc., Oceanside, CA (US)

(21) Appl. No.: 18/179,212

(22) Filed: Mar. 6, 2023

Related U.S. Application Data
(60) Provisional application No. 63/317,505, filed on Mar. 7, 2022.

(51) Int. Cl. A61K 35/28; A61K 31/09; A61K 35/15; A61K 35/17

(52) U.S. Cl. CPC A61K 35/28; A61K 31/09; A61K 35/15; A61K 35/17

(57) ABSTRACT

Disclosed are means, treatments and compositions of matter useful for treatment of chemotherapy/radiotherapy associated cognitive dysfunction. In one embodiment the invention provides the administration of mesenchymal stem cell apoptotic bodies alone or in combination with “regenerative adjuvants” to prevent and/or reverse cognitive dysfunction associated with chemotherapy and/or radiation therapy. In other embodiments the invention teaches the utilization of stem cell apoptotic bodies for induction of neuroregeneration directly or indirectly.

### CROSS-REFERENCE TO RELATED APPLICATIONS

This application claims priority to U.S. Provisional Application No. 63/317,505, filed Mar. 7, 2022, entitled “Treatment of Trauma Associated Cognitive Dysfunction Using Mesenchymal Stem Cell Apoptotic Bodies and Compositions Thereof”, which is incorporated by reference herein in its entirety.

### FIELD OF THE INVENTION

The teachings herein relate to methods and agents for preventing or reducing cognitive decline in a patient following a planned inflammatory trigger in said patient, the method comprising administering a therapeutically effective amount of apoptotic bodies to a patient.

### BACKGROUND

Cognitive decline in patients is a serious and common occurrence. There is a need in the art for improved agents and methods for improving cognitive outcomes.

### SUMMARY

Preferred embodiments are directed to methods for preventing or reducing cognitive decline in a patient following a planned inflammatory trigger in said patient, the method comprising administering a therapeutically effective amount of apoptotic bodies to a patient.

Preferred embodiments are directed to the use of a therapeutically effective amount of apoptotic bodies in the manufacture of a medicament for use in preventing or reducing cognitive decline in a patient following a planned inflammatory trigger in said patient.

Preferred embodiments are directed to an agent for use in preventing or reducing cognitive decline in a patient following a planned inflammatory trigger in said patient, wherein the agent comprises a therapeutically effective amount of apoptotic bodies.

Preferred methods are directed to embodiments wherein the planned inflammatory trigger is surgery and the method, use or agent is for preventing or reducing postoperative cognitive dysfunction (POCD) in said patient.

Preferred methods are directed to embodiments wherein the planned inflammatory trigger is chemotherapy.

Preferred embodiments are directed to methods for reducing cognitive decline in a patient with a cognitive disorder, wherein said patient has been exposed to an inflammatory trigger, the method comprising administering a therapeutically effective amount of apoptotic bodies derived from one or more cell populations to said patient after exposure of said patient to said inflammatory trigger.

Preferred embodiments are directed to methods of using a therapeutically effective amount of apoptotic bodies derived from one or more cell populations in the manufacture of a medicament for use in reducing cognitive decline in a patient with a cognitive disorder, wherein said patient has been exposed to an inflammatory trigger.

Preferred embodiments are directed to an agent for use in reducing cognitive decline in a patient with a cognitive disorder, wherein said patient has been exposed to an inflammatory trigger, and, wherein the agent comprises a therapeutically effective amount of apoptotic bodies derive from one or more cell types.

Preferred methods and agents are directed to embodiments wherein the cognitive disorder is delirium, Alzheimer's Disease, multiple sclerosis, stroke, Parkinson's Disease, Huntington's Disease, dementia, frontotemporal dementia, vascular dementia, HIV dementia, COVID-19 associated dementia, Post-Traumatic Stress Disorder and/or Rheumatoid Arthritis.

Preferred methods and agents are directed to embodiments wherein the inflammatory trigger is infection, trauma, surgery, vaccination, arthritis, obesity, diabetes, stroke, radiation therapy, cardiac arrest, burns, chemotherapy, blast injury, urinary tract infection (UTI), respiratory tract infection (RTI), HIV, poisoning, alcohol or other medication withdrawal, hypoxia, and/or head injury.

Preferred methods are directed to embodiments wherein the POCD is manifested as one or more of memory loss, memory impairment, concentration impairment, delirium, dementia and sickness behaviour.

Preferred methods are directed to embodiments further comprising administering a therapeutically effective amount of immature dendritic cells to said patient.

Preferred methods are directed to embodiments wherein the medicament or agent is for administration in combination with a therapeutically effective amount of immature dendritic cells to said patient.

Preferred methods, uses and agents are directed to embodiments wherein the immature dendritic cell is administered, or is for administration, before, after or simultaneously with apoptotic bodies.

Preferred methods, uses and agents are directed to embodiments wherein the immature dendritic cells is co-administered with the apoptotic bodies.

Preferred methods, uses and agents are directed to embodiments wherein the apoptotic bodies are administered, or is for administration to the patient; before commencement of a surgical procedure; during a surgical procedure; or after completion of a surgical procedure, on said patient.

Preferred methods, uses and agents are directed to embodiments wherein the apoptotic bodies are administered, or is for administration, immediately before or up to 1 hour after completion of said surgical procedure.

Preferred methods, uses and agents are directed to embodiments wherein the apoptotic bodies are administered, or is for administration to the patient; before commencement of chemotherapy; during chemotherapy; or after completion of a round of treatment of chemotherapy on said patient.

Preferred methods, uses and agents are directed to embodiments wherein the patient has, or is at risk of developing, delirium, Alzheimer's Disease, multiple sclerosis, stroke, Parkinson's Disease, Huntington's Disease, dementia, frontotemporal dementia, vascular dementia, HIV dementia, Post-Traumatic Stress Disorder or Rheumatoid Arthritis.

Preferred methods, uses and agents are directed to embodiments wherein the patient is a human.

Preferred methods, uses and agents are directed to embodiments wherein the patient is less than 20 years of age, or over 50 years of age.

Preferred methods, uses and agents are directed to embodiments wherein the apoptotic bodies are derived from peripheral blood mononuclear cells.

Preferred methods, uses and agents are directed to embodiments wherein the apoptotic bodies are derived from a regenerative cell.

Preferred methods, uses and agents are directed to embodiments wherein said apoptotic cells is generated by exposure of cells to one or more agents capable of causing apoptosis.

Preferred methods are directed to embodiments wherein said agent capable of causing apoptosis is ultraviolet irradiation.

Preferred methods are directed to embodiments wherein said agent capable of causing apoptosis is gamma irradiation.

Preferred methods are directed to embodiments wherein said agent capable of causing apoptosis is X-irradiation.

Preferred methods are directed to embodiments wherein said agent capable of causing apoptosis Is UV irradiation together with psoralen.

Preferred methods are directed to embodiments wherein said agent capable of causing apoptosis Is UV irradiation together with porfimer sodium.

Preferred methods are directed to embodiments wherein said agent capable of causing apoptosis is ozone.

Preferred methods are directed to embodiments wherein said agent capable of causing apoptosis is hydrogen peroxide.

Preferred methods, uses and agents are directed to embodiments wherein the apoptotic bodies is for administration, to the patient; before commencement of a surgical procedure; during a surgical procedure; or after completion of a surgical procedure, on said patient.

Preferred methods, uses and agents are directed to embodiments wherein the apoptotic bodies are administered, or is for administration to the patient; before commencement of chemotherapy; during chemotherapy; or after completion of a round of treatment of chemotherapy on said patient.

Preferred methods, uses and agents are directed to embodiments wherein the surgical procedure is a cardiothoracic, an orthopaedic, a neurological, a vascular, a plastic & reconstructive, a gynaecological, an obstetric, a urological, a general, a head & neck, an ear, nose & throat (ENT), a paediatric, a dental, a maxillofacial, an ophthalmic, a pain management, a trauma, or a minor surgical procedure.

Preferred methods, uses and agents are directed to embodiments wherein the general surgical procedure is a colorectal, a hepatobiliary, or an upper gastro-intestinal surgical procedure.

Preferred methods, uses and agents are directed to embodiments wherein the minor surgical procedure is a catheterisation, a minor skin procedure, a minor orthopedic procedure, a nerve block, an endoscopy, a transoesophageal echocardiogram or another minor procedure.

Preferred methods, uses and agents are directed to embodiments wherein the surgical procedure is carried out under general anaesthesia, regional anaesthesia, local anaesthesia, sedation or a combination thereof.

Preferred embodiments are directed to kits of parts comprising apoptotic bodies and immature dendritic cells for use in preventing or reducing cognitive decline in a patient following a planned inflammatory trigger.

Preferred embodiments are directed to kits of parts comprising apoptotic bodies and immature dendritic cells for use in reducing cognitive decline in a patient with a cognitive disorder, wherein said patient has been exposed to an inflammatory trigger.

Preferred embodiments are directed to kits comprising: Apoptotic bodies; Dendritic cells; and instructions for administration of said IL-1 antagoinist and TNFa antagonist to a patient before, during or after a planned inflammatory trigger.

41. Preferred embodiments are directed to kits wherein the planned inflammatory trigger is a surgical procedure or chemotherapy.

Preferred embodiments are directed to methods for preventing or reducing cognitive decline in a patient following a planned inflammatory trigger in said patient, the method comprising administering a therapeutically effective amount of apoptotic bodies to said patient.

Preferred methods are directed to embodiments wherein the planned inflammatory trigger is surgery and the method is for preventing or reducing post-operative cognitive dysfunction (POCD).

Preferred methods are directed to embodiments wherein the planned inflammatory trigger is chemotherapy.

Preferred methods are directed to embodiments for reducing cognitive decline in a patient with a cognitive disorder, wherein said patient has been exposed to an inflammatory trigger, the method comprising administering a therapeutically effective amount of apoptotic bodies to said patient after exposure of said patient to said inflammatory trigger.

Preferred methods are directed to embodiments wherein the cognitive disorder is delirium, Alzheimer's Disease, multiple sclerosis, stroke, Parkinson's Disease, Huntington's Disease, dementia, frontotemporal dementia, vascular dementia, HIV dementia, Post-Traumatic Stress Disorder and/or Rheumatoid Arthritis.

Preferred methods are directed to embodiments wherein the inflammatory trigger is infection, trauma, surgery, vaccination, arthritis, obesity, diabetes, stroke, cardiac arrest, burns, chemotherapy, blast injury, urinary tract infection (UTI), respiratory tract infection (RTI), HIV, poisoning, alcohol or other medication withdrawal, hypoxia, and/or head injury.

Preferred methods are directed to embodiments wherein the POCD is manifested as one or more of memory loss, memory impairment, concentration impairment, delirium, dementia and sickness behaviour.

Preferred methods are directed to embodiments wherein the patient has, or is at risk of developing, delirium, Alzheimer's Disease, multiple sclerosis, stroke, Parkinson's Disease, Huntington's Disease, dementia, frontotemporal dementia, vascular dementia, HIV dementia, Post-Traumatic Stress Disorder and/or Rheumatoid Arthritis.

Preferred methods are directed to embodiments wherein the patient is a human.

Preferred methods are directed to embodiments wherein the patient is less than 20 years of age or over 50 years of age.

Preferred methods are directed to embodiments wherein the apoptotic bodies are derived from umbilical cord mesenchymal stem cells and immature dendritic cells are generated from peripheral blood.

Preferred methods are directed to embodiments wherein the apoptotic bodies are generated by treatment of mesenchymal stem cells with ozone.

Preferred methods are directed to embodiments wherein the ozone gas is administered at 5 micrograms of ozone per milliliter of medical grade oxygen.

Preferred methods are directed to embodiments wherein the ozone gas is administered at 1 micrograms of ozone per milliliter of medical grade oxygen.

Preferred methods are directed to embodiments wherein the apoptotic bodies are administered to the patient; before commencement of a surgical procedure; during a surgical procedure; or after completion of a surgical procedure, on said patient.

Preferred methods are directed to embodiments wherein the surgical procedure is a cardiothoracic, an orthopaedic, a neurological, a vascular, a plastic & reconstructive, a gynaecological, an obstetric, a urological, a general, a head & neck, an ear, nose & throat (ENT), a paediatric, a dental, a maxillofacial, an ophthalmic, a pain management, a trauma, or a minor surgical procedure.

Preferred methods are directed to embodiments wherein the general surgical procedure is a colorectal, a hepatobiliary, or an upper gastro-intestinal surgical procedure.

Preferred methods are directed to embodiments wherein the minor surgical procedure is a catheterisation, a minor skin procedure, a minor orthopedic procedure, a nerve block, an endoscopy, a transoesophageal echocardiogram or another minor procedure.

Preferred methods are directed to embodiments wherein the surgical procedure is carried out under general anaesthesia, regional anaesthesia, local anaesthesia, sedation or a combination thereof.

Preferred methods are directed to embodiments wherein said apoptotic bodies derived from a regenerative cell.

Preferred methods are directed to embodiments wherein said apoptotic bodies are administered together with a Sertoli cell and/or apoptotic bodies derived from a Sertoli cell.

Preferred methods are directed to embodiments wherein said regenerative cells are activated prior to induction to undergo apoptosis.

Preferred methods are directed to embodiments wherein said regenerative cells are activated by contact with allogeneic T cells.

Preferred methods are directed to embodiments wherein said regenerative cells are activated by culture with an inflammatory cytokine.

Preferred methods are directed to embodiments wherein said regenerative cells are activated by culture with an activator of NF-kappa B.

Preferred methods are directed to embodiments wherein said regenerative cells are activated by culture with an activator of the JAK-S TAT pathway.

Preferred methods are directed to embodiments wherein said regenerative cells are activated by culture with an activator of NF-kappa B.

Preferred methods are directed to embodiments wherein said regenerative cells are activated by culture with an activator of the toll like receptor pathway.

Preferred methods are directed to embodiments wherein said regenerative cells are activated by culture with an activator of the retinoic acid inducible gene.

Preferred methods are directed to embodiments wherein said regenerative cells are activated by culture with an activator of the melanoma differentiation associated gene-5.

Preferred methods are directed to embodiments wherein said regenerative cells are activated by culture with an activator of the nucleotide-binding oligomerization domain-containing protein.

Preferred methods are directed to embodiments wherein said regenerative cells are activated by treatment with interleukin-1.

Preferred methods are directed to embodiments wherein said regenerative cells are activated by treatment with interferon alpha.

Preferred methods are directed to embodiments wherein said regenerative cells are activated by treatment with interferon beta.

Preferred methods are directed to embodiments wherein said regenerative cells are activated by treatment with interferon gamma.

Preferred methods are directed to embodiments wherein said regenerative cells are activated by treatment with interferon omega.

Preferred methods are directed to embodiments wherein said regenerative cells are activated by treatment with Poly IC.

Preferred methods are directed to embodiments wherein said regenerative cells are activated by treatment with lipopolysaccharide.

Preferred methods are directed to embodiments wherein said regenerative cells are activated by treatment with low molecular weight hyaluronic acid.

Preferred methods are directed to embodiments wherein said regenerative cells are activated by treatment with CpG motifs.

Preferred methods are directed to embodiments wherein said regenerative cells are activated by treatment with neutrophil extracellular traps.

Preferred methods are directed to embodiments wherein said regenerative cell is a stem cell.

Preferred methods are directed to embodiments wherein said stem cell is a hematopoietic stem cell.

Preferred methods are directed to embodiments wherein said hematopoietic stem cell is capable of generating leukocytic, lymphocytic, thrombocytic and erythrocytic cells when transplanted into an immunodeficient animal.

Preferred methods are directed to embodiments wherein said hematopoietic stem cell is non-adherent to plastic.

Preferred methods are directed to embodiments wherein said hematopoietic stem cell is adherent to plastic.

Preferred methods are directed to embodiments wherein said hematopoietic stem cell is exposed to hyperthermia.

Preferred methods are directed to embodiments wherein said hematopoietic stem cell expresses interleukin-3 receptor.

Preferred methods are directed to embodiments wherein said hematopoietic stem cell expresses interleukin-1 receptor.

Preferred methods are directed to embodiments wherein said hematopoietic stem cell expresses c-met.

Preferred methods are directed to embodiments wherein said hematopoietic stem cell expresses mpl.

Preferred methods are directed to embodiments wherein said hematopoietic stem cell expresses interleukin-11 receptor.

Preferred methods are directed to embodiments wherein said hematopoietic stem cell expresses G-CSF receptor.

Preferred methods are directed to embodiments wherein said hematopoietic stem cell expresses GM-CSF receptor.

Preferred methods are directed to embodiments wherein said hematopoietic stem cell expresses M-CSF receptor.

Preferred methods are directed to embodiments wherein said hematopoietic stem cell expresses VEGF-receptor.

Preferred methods are directed to embodiments wherein said hematopoietic stem cell expresses c-kit.

Preferred methods are directed to embodiments wherein said hematopoietic stem cell expresses CD33.

Preferred methods are directed to embodiments wherein said hematopoietic stem cell expresses CD133.

Preferred methods are directed to embodiments wherein said hematopoietic stem cell expresses CD34.

Preferred methods are directed to embodiments wherein said hematopoietic stem cell expresses Fas ligand.

Preferred methods are directed to embodiments wherein said hematopoietic stem cell does not express lineage markers.

Preferred methods are directed to embodiments wherein said hematopoietic stem cell does not express CD14.

Preferred methods are directed to embodiments wherein said hematopoietic stem cell does not express CD16.

Preferred methods are directed to embodiments wherein said hematopoietic stem cell does not express CD3.

Preferred methods are directed to embodiments wherein said hematopoietic stem cell does not express CD56.

Preferred methods are directed to embodiments wherein said hematopoietic stem cell does not express CD38.

Preferred methods are directed to embodiments wherein said hematopoietic stem cell does not express CD30.

Preferred methods are directed to embodiments wherein said regenerative cell is a mesenchymal stem cell.

Preferred methods are directed to embodiments wherein said mesenchymal stem cells are naturally occurring mesenchymal stem cells.

Preferred methods are directed to embodiments wherein said mesenchymal stem cells are generated in vitro.

Preferred methods are directed to embodiments wherein said naturally occurring mesenchymal stem cells are tissue derived.

Preferred methods are directed to embodiments wherein said naturally occurring mesenchymal stem cells are derived from a bodily fluid.

Preferred methods are directed to embodiments wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; 1) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred methods are directed to embodiments wherein said tissue derived mesenchymal stem cells are isolated from tissues containing cells selected from a group of cells comprising of: endothelial cells, epithelial cells, dermal cells, endodermal cells, mesodermal cells, stems, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue stems, corneal keratocytes, tendon stems, bone marrow reticular tissue stems, nonepithelial stems, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Preferred methods are directed to embodiments wherein said mesenchymal stem cells are plastic adherent.

Preferred methods are directed to embodiments wherein said mesenchymal stem cells express a marker selected from a group comprising of: a) CD73; b) CD90; and c) CD105.

Preferred methods are directed to embodiments wherein said mesenchymal stem cells lack expression of a marker selected from a group comprising of: a) CD14; b) CD45; and c) CD34.

Preferred methods are directed to embodiments wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Preferred methods are directed to embodiments wherein said mesenchymal stem cells from umbilical cord tissue do not express markers selected from a group comprising of: a) CD117; b) CD31; c) CD34; and CD45;

Preferred methods are directed to embodiments wherein said mesenchymal stem cells from umbilical cord tissue express, relative to a human stem, increased levels of interleukin 8 and reticulon 1

Preferred methods are directed to embodiments wherein said mesenchymal stem cells from umbilical cord tissue have the potential to differentiate into cells of at least a skeletal muscle, vascular smooth muscle, pericyte or vascular endothelium phenotype.

Preferred methods are directed to embodiments wherein said mesenchymal stem cells from umbilical cord tissue express markers selected from a group comprising of: a) CD10; b) CD13; c) CD44; d) CD73; and e) CD90.

Preferred methods are directed to embodiments wherein said umbilical cord tissue mesenchymal stem cell is an isolated umbilical cord tissue cell isolated from umbilical cord tissue substantially free of blood that is capable of self-renewal and expansion in culture,

Preferred methods are directed to embodiments wherein said umbilical cord tissue mesenchymal stem cells has the potential to differentiate into cells of other phenotypes.

Preferred methods are directed to embodiments wherein said other phenotypes comprise: a) osteocytic; b) adipogenic; and c) chondrogenic differentiation.

Preferred methods are directed to embodiments wherein said cord tissue derived mesenchymal stem cells can undergo at least 20 doublings in culture.

Preferred methods are directed to embodiments wherein said cord tissue derived mesenchymal stem cell maintains a normal karyotype upon passaging

Preferred methods are directed to embodiments wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Preferred methods are directed to embodiments wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Preferred methods are directed to embodiments wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; 1) RANTES; and m) TIMP1

Preferred methods are directed to embodiments wherein said umbilical cord tissue derived cells express markers selected from a group comprising of: a) TRA1-60; b) TRA1-81; c) SSEA3; d) SSEA4; and e) NANOG.

Preferred methods are directed to embodiments wherein said umbilical cord tissue-derived cells are positive for alkaline phosphatase staining.

Preferred methods are directed to embodiments wherein said cognitive decline is associated with COVID-19.

Preferred methods are directed to embodiments wherein said cognitive decline is associated with chronic traumatic encephalopathy.

Preferred methods are directed to embodiments wherein said cognitive decline is associated with brain injury.

### BRIEF DESCRIPTION OF THE DRAWINGS

FIG. 1 is a bar graph showing interleukin-1 levels in mice injected with: control, peterostilebene, and apoptotic bodies.

### DETAILED DESCRIPTION OF THE INVENTION

The invention teaches the use of apoptotic bodies, in particular embodiments apoptotic bodies from mesenchymal stem cells, as a treatment for cognitive dysfunctions. In one particular embodiment, the invention teaches the utilization of apoptotic bodies as a means of treatment of delirium and other symptoms of cognitive disorders have been associated with chemotherapy, radiotherapy or other treatment of cancer patients. Thus, it is envisaged that the present invention may be utilized to prevent or reduce cognitive decline in cancer patients following chemotherapy and/or radiotherapy treatment, either prophylactically, or as a treatment.

For the purpose of the invention, “cognitive decline” includes the meaning of any deterioration of cognitive function brought about by a cognitive disorder and/or an inflammatory trigger. Cognitive decline has been previously associated with inflammation and the practitioner of the invention is referred to the following publications with are incorporated by reference. Examples of inflammation associated cognitive decline include: a) Chemotherapy associated cognitive decline [1]; b) hyperhomocystinemia associated cognitive decline [2]; c) fatigue associated cognitive decline [3]; d) lung injury associated cognitive decline [4], HIV associated cognitive decline [5, 6], sepsis associated cognitive impairment [7-9]; e) autoimmune arthritis associated cognitive impairment [10]; f) metal associated cognitive impairment [11]; g) ovariectomy induced cognitive impairment [12]; h) diabetes associated cognitive decline [13];

For the purpose of the invention, “post-operative cognitive dysfunction”, includes the deterioration of intellectual function reflected as memory and concentration impairment presenting in a patient after that patient has undergone a surgical procedure. Some examples of POCD are listed in the following publications and incorporated by reference [14, 15].

Such deterioration of intellectual function may take many forms and as such this definition includes any form of cognitive decline presenting post-operatively. The present invention is considered to be particularly useful when administered before, during or immediately following surgery. In general, cognitive dysfunctions following surgery are common and effective immediately following recovery. Classical POCD characterises a more prolonged and subtle dysfunction in cognitive domains, juxtaposed to a more evident but short-lived “delirium” (both are included in the above definition of POCD). Discrimination between cognitive dysfunctions is made in particular according to the length of the cognitive impairment; delirium resolves itself usually after few days, whereas POCD persists for months (>3) and can become a permanent dysfunction. Thus, such cognitive decline falling within the scope of the above definition may be short-lived, thus may ablate hours or days after completion of the surgical procedure; or the cognitive decline may persist over the course of months or years, or the cognitive decline may even be permanent. Delirium is commonly seen after surgery, usually soon after surgery (hours to days) and fluctuating over time. Although the dysfunction lasts over a short period of time, delirium is associated with increased mortality (Ely et al. 2004), greater care dependency, costs (Milbrandt et al. 2004) and prolonged hospitalization (Ely et al. 2001). It is considered that the use of the present invention will aid in reducing or preventing this deterioration of intellectual function and lead to an improvement in the quality of life of the patient and his/her carers.

The diagnosis of POCD may be aided by neuropsychological testing. In general, the presence of POCD may be suspected when memory loss is greater than expected under normal situations. At present, there are no specific cognitive sets for successful POCD diagnosis; generally multiple neurocognitive assessments are made before reaching a diagnosis. It is envisaged that the symptoms of POCD may include memory loss, memory impairment, concentration impairment, delirium, dementia, and/or sickness behaviour.

By “delirium” is included an acute and debilitating decline in attention, focus, perception, and cognition that produces an altered form of semi-consciousness. Delirium is a syndrome, or group of symptoms, caused by a disturbance in the normal functioning of the brain. The delirious patient has a reduced awareness of and responsiveness to the environment, which may be manifested as disorientation, incoherence, and memory disturbance. Delirium affects at least one in 10 hospitalised patients, and 1 in 2 elderly hospitalised patients. Whilst it is not a specific disease itself, patients with delirium usually fare worse than those with the same illness who do not have delirium. It occurs as a post-operative complication, with evidence from the mouse model described in the Examples showing that it can be caused by an inflammatory trigger. This would also explain why delirium is seen in patients admitted to hospital as a result of other inflammatory triggers, for example, stroke (CVA), Heart Attack (MI), urinary tract infection (UTI), respiratory tract infection (RTI), poisoning, alcohol or other medication withdrawal, hypoxia, and head injury.

By “dementia” we mean a serious cognitive disorder, which may be static, the result of a unique global brain injury or progressive, resulting in long-term decline in cognitive function due to damage or disease in the body beyond what might be expected from normal aging.

By “sickness behaviour” are included symptoms ranging from lethargy, fever, decreased food intake, somnolence, hyperalgesia, and general fatigue to social withdrawal and memory impairment (Dantzer R: Cytokine-induced sickness behaviour: a neuroimmune response to activation of innate immunity. Eur J Pharmacol 2004, 500(1-3):399-411).

In some embodiments of the invention, apoptotic bodies are administered together with histone deacetylase inhibitors in order to increase neural plasticity and therefore reduce cognitive impairment. The utilization of sodium butyrate for improvement of neural plasticity has been described and is incorporated by reference [16-24].

The present inventors have demonstrated that microglial activation and associated inflammation are associated with the onset of POCD. Further, the ablation of microglial activation with minocycline was found to prevent post-operative memory loss in the in vivo models used. Thus, a method for assessing the onset of, or the progress of treatment for, POCD may be the analysis of microglial activation in the brain of the patient. Such activation may be measured using techniques such as Positron Emission Tomography (PET) scanning of the patient's brain. Such PET scanning may, for example, be conducted using .sup.11C-PK11195, which is a ligand for the peripheral benzodiazepine receptor. An elevation of microglial activation may be an indication of POCD (or vice versa). Further methods of assessing POCD may include magnetic resonance imaging (MRI) or PET with FEPPA or .sup.11C-PK11195. Other imaging techniques including MRI with diffusion tensor imaging and MR spectroscopy can also be used to non-invasively assess POCD. Risk factors for the development of POCD include advanced age in the patient, the patient's level of education and “cognitive reserve”, potential genetic polymorphisms (for example APOe4) and co-morbidities, such as underlying neurological disease.

By “preventing POCD” we include the meaning that the method, use or agent of the invention is considered to reduce the likelihood of the occurrence of POCD in a patient who has undergone a surgical procedure. Thus, the invention may be used, or be for use, prophylactically before any sign of POCD develops in the patient. While it is preferred that POCD is prevented from occurring in the patient, it is understood that some incidence of POCD may still remain but it is envisaged that the use of the present invention will reduce the symptoms of, and/or reduce the persistence of, that POCD. Thus, by “reducing POCD” we include the meaning that the onset of POCD is lessened or delayed and the symptoms are reduced thus improving the cognition of the patient while perhaps not entirely preventing the onset of the POCD. This can be established by a battery of neuropsychological tests. The invention may also be used following presentation of POCD in a patient, as a treatment for the POCD.

By “cognitive disorder” we include the meaning of any neurological disease, condition or disorder that manifests in impaired cognitive function in a patient. Such disorders may arise in patients of any age. The symptoms of such disorders may include drowsiness, fatigue, concentration impairment, vertigo, confusion, memory impairment, memory loss, delirium, loss of motor neurone control and other such symptoms as would be understood by a person of skill in the art. As explained above, delirium is a symptom, or group of symptoms, but is also a syndrome, which is caused by a disturbance in the normal functioning of the brain. Thus, it is envisaged that while delirium may be a symptom of the cognitive disorder, where another disorder is present, it may also be the only cognitive disorder that has presented in the patient and thus the present invention may be beneficial where no other cognitive disorder has yet been characterised but the patient is exhibiting signs of delirium. Further examples of cognitive disorders encompassed herein include Alzheimer's Disease, multiple sclerosis, stroke, Parkinson's Disease, Huntington's Disease, dementia, frontotemporal dementia, vascular dementia, HIV dementia, Post-Traumatic Stress Disorder and chronic inflammatory conditions such as Rheumatoid Arthritis. Further examples of relevant conditions would be known to the skilled person.

“inflammatory trigger” we include the meaning of any insult to the body that results in an inflammatory response. Such an inflammatory response, if left unchecked, may lead to an overactive neuroinflammatory response and cause or worsen (if a cognitive disorder is already present) the cognitive condition of patients. A non-exhaustive list of examples of such inflammatory triggers includes infection, trauma (such as broken bones after a fall), surgery, vaccination, arthritis, obesity, diabetes, stroke (CVA), cardiac arrest (heart attack; myocardial infarction (MI)), burns, chemotherapy, blast injury, urinary tract infection (UTI), respiratory tract infection (RTI), Human immunodeficiency virus infection (HIV), poisoning, alcohol or other medication withdrawal, hypoxia, and head injury.

It is envisaged that the patient, while potentially not already having been diagnosed with a cognitive disorder, may be at risk of developing a cognitive disorder. Thus, the present invention may also be beneficial to patients who are at risk of developing a cognitive disorder. Such patients may include the elderly or individuals who have a familial history of such disorders.

Preferred unit dosage formulations are those containing a daily dose or unit, daily sub-dose or an appropriate fraction thereof, of an active ingredient. It is preferred that doses for topical administration of the antagonists of the invention may be of the order of fractions of or multiple mg/kg body weight of the patient. For example, the dose may be between 0.01 to 500 mg/kg body weight; 1 to 400 mg/kg body weight; 2 to 200 mg/kg body weight; 3 to 100 mg/kg body weight or 4 to 50 mg/kg (or any combination of these upper and lower limits, as would be appreciated by the skilled person). The dose used may in practice be limited by the solubility of the compound. Examples of possible doses are 0.01, 0.05, 0.075, 0.1, 0.2, 0.5, 0.7, 1, 2, 5, 10, 12, 15, 20, 25, 30, 35, 40, 45, 50 or 100 mg per kg body weight up to, for example 500 mg/kg body weight, or any value in between. It is envisaged that preferred doses of antagonist would be adjusted according to relative potency. The physician or veterinary practitioner will be able to determine the required dose in a given situation based on the teaching and Examples provided herein.

In some embodiments of the invention, hydrogen gas is administered together with apoptotic bodies in order to increase ability of hydrogen gas to reduce cognitive decline [25].

### Example 1: Reduction of LPS Induced Inflammation

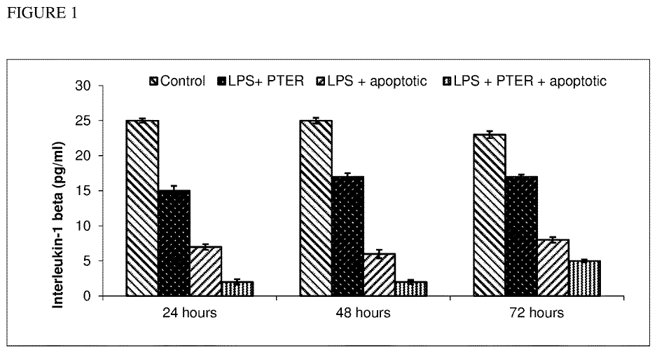

BALB/c mice were treated according to the figure below: Control was saline injection, LPS was administered intraperitoneally at 1 microgram per mouse, peterostilebene was administered at 10 micrograms per mouse, and apoptotic bodies at 1 microgram per mouse. Apoptotic bodies were purified from hydrogen peroxide treated JadiCells. Mice (10 per group) were sacrificed and interleukin-1 was assessed in plasma using ELISA. Results are shown in FIG. 1.

### REFERENCES

1. Janelsins, M. C., et al., Relationships between cytokines and cognitive function from pre-to post-chemotherapy in patients with breast cancer. J Neuroimmunol, 2022. 362: p. 577769.
2. Verma, N., et al., Ameliorative Effect of Acetyl L-carnitine in Alzheimer's Disease via Downregulating of Homocysteine Levels in Hyperhomocysteinemia Induced Cognitive Deficit in Mouse Model. Drug Metab Lett, 2021. 14(3): p. 219-231.
3. Kozlova, E. V., et al., Induction of distinct neuroinflammatory markers and gut dysbiosis by differential pyridostigmine bromide dosing in a chronic mouse model of GWI showing persistent exercise fatigue and cognitive impairment. Life Sci, 2022. 288: p. 120153.
4. Wang, X., et al., Dimethyl fumarate prevents acute lung injury related cognitive impairment potentially via reducing inflammation. J Cardiothorac Surg, 2021. 16(1): p. 331.
5. Lee, Y. J., et al., Amyloidogenic, neuroinflammatory and memory dysfunction effects of HIV-1 gp120. Arch Pharm Res, 2021. 44(7): p. 689-701.
6. Ruhanya, V., et al., Plasma Cytokine Levels As Predictors of Global and Domain-Specific Human Immunodeficiency Virus-Associated Neurocognitive Impairment in Treatment-Naive Individuals. J Interferon Cytokine Res, 2021. 41(4): p. 153-160.
7. Zhang, N., et al., Protective effects and mechanisms of high-dose vitamin C on sepsis-associated cognitive impairment in rats. Sci Rep, 2021. 11(1): p. 14511.
8. Chataigner, M., et al., Fish Hydrolysate Supplementation Containing n-3 Long Chain Polyunsaturated Fatty Acids and Peptides Prevents LPS-Induced Neuroinflammation. Nutrients, 2021. 13(3).
9. Jin, P., et al., INT-777 prevents cognitive impairment by activating Takeda G protein-coupled receptor 5 (TGR5) and attenuating neuroinflammation via cAMP/PKA/CREB signaling axis in a rat model of sepsis. Exp Neurol, 2021. 335: p. 113504.
10. Poutoglidou, F., et al., Infliximab and tocilizumab reduce anxiety-like behavior, improve cognitive performance and reverse neuropathological alterations in juvenile rats with severe autoimmune arthritis. Int Immunopharmacol, 2021. 99: p. 107917.
11. Kaur, S., et al., Allicin ameliorates aluminium-and copper-induced cognitive dysfunction in Wistar rats: relevance to neuro-inflammation, neurotransmitters and Abeta (1-42) analysis. J Biol Inorg Chem, 2021. 26(4): p. 495-510.
12. Zheng, X., et al., Protective effects of Lycium barbarum polysaccharide on ovariectomyinduced cognition reduction in aging mice. Int J Mol Med, 2021. 48(1).
13. Xianchu, L., et al., Apocynin ameliorates cognitive deficits in streptozotocin-induced diabetic rats. Bratisl Lek Listy, 2021. 122(1): p. 78-84.
14. Lee, T. A., et al., Assessment of the emergence of Alzheimer's disease following coronary artery bypass graft surgery or percutaneous transluminal coronary angioplasty. J Alzheimers Dis, 2005. 7(4): p. 319-24.
15. Bitsch, M. S., et al., Acute cognitive dysfunction after hip fracture: frequency and risk factors in an optimized, multimodal, rehabilitation program. Acta Anaesthesiol Scand, 2006. 50(4): p. 428-36.
16. Jiang, Y., et al., Sodium butyrate ameliorates the impairment of synaptic plasticity by inhibiting the neuroinflammation in 5XFAD mice. Chem Biol Interact, 2021. 341: p. 109452.
17. Kitahara, M., et al., Exercise and pharmacological inhibition of histone deacetylase improves cognitive function accompanied by an increase of gene expressions crucial for neuronal plasticity in the hippocampus. Neurosci Lett, 2021. 749: p. 135749.
18. Chen, G., et al., A large dose of methamphetamine inhibits drugevoked synaptic plasticity via ER stress in the hippocampus. Mol Med Rep, 2021. 23(4).
19. Maejima, H., et al., Effects of exercise and pharmacological inhibition of histone deacetylases (HDACs) on epigenetic regulations and gene expressions crucial for neuronal plasticity in the motor cortex. Brain Res, 2021. 1751: p. 147191.
20. Zuzina, A. B., A. K. Vinarskaya, and P. M. Balaban, Histone deacetylase inhibitors rescue the impaired memory in terrestrial snails. J Comp Physiol A Neuroethol Sens Neural Behav Physiol, 2020. 206(4): p. 639-649.
21. Drissi, I., et al., Memory and plasticity impairment after binge drinking in adolescent rat hippocampus: GluN2A/GluN2B NMDA receptor subunits imbalance through HDAC2.

Addict Biol, 2020. 25(3): p. e12760.
22. Yoo, D. Y., et al., Sodium butyrate, a histone deacetylase Inhibitor, ameliorates SIRT2-induced memory impairment, reduction of cell proliferation, and neuroblast differentiation in the dentate gyrus. Neurol Res, 2015. 37(1): p. 69-76.
23. Dash, P. K., S. A. Orsi, and A. N. Moore, Histone deactylase inhibition combined with behavioral therapy enhances learning and memory following traumatic brain injury. Neuroscience, 2009. 163(1): p. 1-8.
24. Morita, K., et al., Histone deacetylase inhibitors promote neurosteroid-mediated cell differentiation and enhance serotonin-stimulated brain-derived neurotrophic factor gene expression in rat C6 glioma cells. J Neurosci Res, 2009. 87(11): p. 2608-14.
25. Jeong, E. S., et al., Therapeutic Effects of Hydrogen Gas Inhalation on Trimethyltin-Induced Neurotoxicity and Cognitive Impairment in the C57BL/6 Mice Model. Int J Mol Sci, 2021. 22(24).

1. A method for preventing or reducing cognitive decline in a patient following an inflammatory trigger in said patient, the method comprising administering a therapeutically effective amount of apoptotic bodies to a patient alone or together with pterostilbene or an analogue thereof.

2. The method of claim 1, wherein said inflammatory trigger is selected from a group comprising of: a) exposure to a drug of abuse known to activate TLR4 signaling; b) exposure to COVID-19 or a vaccine for said COVID-19; c) traumatic brain injury; d) chronic traumatic encephalopathy; and e) surgery.

3. The method of claim 1, wherein said cognitive disorder is selected from a group comprising of: a) delirium; b) Alzheimer's Disease; c) multiple sclerosis; d) stroke; e) Parkinson's Disease; f) Huntington's Disease; g) dementia; h) frontotemporal dementia; i) vascular dementia, j) HIV dementia; and k) Post-Traumatic Stress Disorder.

4. The method of claim 1, wherein said inflammatory trigger is infection, trauma, surgery, vaccination, arthritis, obesity, diabetes, stroke, radiation therapy, cardiac arrest, burns, chemotherapy, blast injury, urinary tract infection (UTI), respiratory tract infection (RTI), HIV, poisoning, alcohol or other medication withdrawal, hypoxia, and/or head injury.

5. The method of claim 1, wherein said apoptotic bodies are derived from peripheral blood mononuclear cells.

6. The method of claim 1, wherein said apoptotic bodies are derived from monocytes.

7. The method of claim 1, wherein said apoptotic bodies are derived from T regulatory cells.

8. The method of claim 6, wherein said monocytes express CD14.

9. The method of claim 6, wherein said monocytes express CD56.

10. The method of claim 6, wherein said monocytes are M2 monocytes.

11. The method of claim 1, wherein said apoptotic bodies are derived from a regenerative cell.

12. The method of claim 11, wherein said regenerative cell is a mesenchymal stem cell.

13. The method of claim 12, wherein said mesenchymal stem cell expresses CD56.

14. The method of claim 12, wherein said mesenchymal stem cell expresses CD105.

15. The method of claim 12, wherein said mesenchymal stem cell expresses BDNF receptor.

16. The method of claim 1, wherein said apoptotic bodies are generated by exposure of cells to one or more agents capable of causing apoptosis.

17. The method of claim 16, wherein said agent capable of causing apoptosis is ultraviolet irradiation and/or exposure to ozone gas, and/or exposure to hydrogen peroxide.

18. The method of claim 16, wherein said agent capable of causing apoptosis is gamma irradiation, x-irradiation, alpha irradiation or beta irradiation.

19. The method of claim 1, wherein said patient is treated with apoptotic bodies derived from 10,000 to 15,000,000 umbilical cord mesenchymal stem cell cells per kilogram of patient body weight together with 10 ug to 2 grams of pterostilbene per kilogram of patient body weight.

20. The method of claim 19, wherein said apoptotic bodies are generated by exposure to a sufficient concentration of hydrogen peroxide to induce apoptosis.
